(19) United States
(12) Patent Application Publication
(10) Pub. No.: US 2024/0254394 A1
(43) Pub. Date: Aug. 1, 2024

(54) SYSTEM FOR CONDUCTING HIGH-TEMPERTURE THERMOLYSIS OF WASTE MIXTURE

(71) Applicant: IFALLIANCEUSA LLC, New York, NY (US)

(72) Inventor: Farhad MAMMADOV, BRICK, NJ (US)

(21) Appl. No.: 18/103,693

(22) Filed: Jan. 31, 2023

(51) Int. Cl. C10B 49/08; B02C 19/00; B09B 3/35; B09B 3/40; C01B 3/02; C02F 11/10; C07C 1/04; C09C 1/48; C10B 53/02; C10B 57/10

(52) U.S. Cl. CPC C10B 49/08; B02C 19/0031; B09B 3/35; B09B 3/40; C01B 3/02; C02F 11/10; C07C 1/0485; C09C 1/48; C10B 53/02; C10B 57/10; B09B 2101/85; C02F 2303/26

(57) ABSTRACT

A system for conducting high-temperature thermolysis of a waste mixture formed by sewage sludge and wood waste (e.g., creosote-impregnated wooden railway sleepers and utility poles) is proposed. The products of the high-temperature thermolysis may be used to produce thermal energy, electrical energy, carbon black, and liquid fractions which may be used profitably for various purposes.

### TECHNICAL FIELD

The present disclosure relates generally to the field of thermal destruction of solid industrial wastes. In particular, the present disclosure relates to a system for conducting high-temperature thermolysis of a waste mixture (e.g., formed by sewage sludge, creosote-impregnated wooden railway sleepers and utility poles, etc.) to produce thermal energy, electrical energy, carbon black, and liquid fractions.

### BACKGROUND

The growth of the urban population and the development of industry are accompanied by an increase in the volume of wastewater and their sewage sludge. In industrialized countries, each inhabitant generates, on average, 19-25 kg of dry sewage sludge per year, which leads to the accumulation of a large amount of sewage sludge. The sewage sludge belongs to an industrial waste to be recycled. The problem of recycling the sewage sludge is relevant, and a selected recycling scheme should provide high-quality recycling aimed at producing thermal energy, electrical energy, carbon black and liquid fractions which are subject to further beneficial use.

End-of-life creosote-impregnated wooden railway sleepers and utility poles are industrial wastes that must be recycled to produce fuel. In the construction of railway tracks, wooden sleepers were used in the past, which had to be protected from rapid decay. For this purpose, a special antiseptic, mainly creosote, was used. This substance is very toxic, and, therefore, the storage of the end-of-life wooden sleepers and utility poles is the reason for a large concentration of toxins and poisonous gases in the place of their storage. Creosote (coal creosote oil) is a product of the distillation of coal tar at a temperature of 200 to 400° C. It is a dark brown liquid (having a specific gravity of 1.05 to 1.10 g/cm3 and a boiling point of 180 to) 200° ° C. with a pungent odor. Creosote contains 20.1% phenols, 17.2% phenanthrenes, 16.9% pyrenes, 22% acetone and 12% butanol. These compounds, once in the air, can cause severe poisoning in humans and the appearance of cancer diseases.

Thus, there is a need for a technical solution that enables recycling of the sewage sludge and the creosote-impregnated wooden railway sleepers and utility poles.

### SUMMARY

This summary is provided to introduce a selection of concepts in a simplified form that are further described below in the detailed description. This summary is not intended to identify key features of the present disclosure, nor is it intended to be used to limit the scope of the present disclosure.

It is an objective of the present disclosure to provide a technical solution that enables recycling of sewage sludge and wooden waste (e.g., creosote-impregnated wooden railway sleepers and utility poles) by means of high-temperature thermolysis.

The objective above is achieved by the features of the independent claim in the appended claims. Further embodiments and examples are apparent from the dependent claims, the detailed description, and the accompanying drawings.

According to an aspect, a system for conducting high-temperature thermolysis of a waste mixture is provided. The system comprises a sewage sludge storage configured to receive and store sewage sludge, and a wood waste storage configured to receive and store wood waste. The system further comprises a grinding unit coupled to the wood waste storage and configured to grind the wood waste, and a mixer configured to form the waste mixture by mixing the sewage sludge from the sewage sludge storage and the grinded wood waste from the grinding unit. The system further comprises a screw-conveyor dryer configured to heat and dehumidify the waste mixture, and a metering hopper arranged between the mixer and the screw-conveyor dryer. The metering hopper is configured to perform a metered supply of the waste mixture from the mixer to the screw-conveyor dryer while replacing ambient air with carbon dioxide (CO₂). The system further comprises a striker mill coupled to the screw-conveyor dryer. The striker mill is configured to conduct the high-temperature thermolysis of the waste mixture and regrind the waste mixture. The system further comprises a cyclonic separator coupled to the striker mill and configured to separate the waste mixture subjected to the high-temperature thermolysis and regrinding into a solid fraction and a gaseous fraction. The solid fraction of the waste mixture comprises carbon black. The system further comprises a carbon black storage coupled to the cyclonic separator and configured to receive and store the carbon black from the waste mixture. The system further comprises a refrigerator-type condenser coupled to the cyclonic separator and configured to obtain a thermolysis liquid and a synthesis gas by cooling and condensing the gaseous fraction of the waste mixture. The system further comprises a first thermolysis liquid storage coupled to the refrigerator-type condenser and configured to receive and store the thermolysis liquid. The system further comprises a first synthesis gas storage coupled to the refrigerator-type condenser and configured to receive and store the synthesis gas. The system further comprises a second synthesis gas storage coupled to the first synthesis gas storage, and a gas compressor arranged between the first synthesis gas storage and the second synthesis gas storage. The gas compressor is configured to pump over the synthesis gas from the first synthesis gas storage to the second synthesis gas storage while increasing a pressure of the synthesis gas up to 12 atm. The system further comprises a second thermolysis liquid storage coupled to the gas compressor and configured to collect and store a secondary thermolysis liquid resulted from compressing the synthesis gas by the gas compressor when pumping over the synthesis gas from the first synthesis gas storage to the second synthesis gas storage. The system further comprises a fine gas filter coupled to the second synthesis gas storage and configured to clean the synthesis gas from mechanical impurities. The system further comprises a gas holder coupled to the fine gas filter and configured to receive and store the synthesis gas under pressure of up to 30 atm, and an energy conversion unit coupled to the gas holder and configured to convert chemical energy stored in the pressurized synthesis gas into thermal energy and electrical energy. By using the system thus configured, any sewage sludge and any wood waste may be recycled jointly and efficiently to produce thermal energy, electrical energy, carbon black, and liquid fractions which may be used profitably for various purposes.

In one embodiment, the grinding unit comprises a first grinding subunit, a second grinding subunit and a third grinding subunit which are arranged in sequence between the wood waste storage and the mixer. The first grinding subunit is configured to grind the wood waste to coarse wood-waste fractions ranging in size from 100 mm to 200 mm. The second grinding subunit is configured to grind the coarse wood-waste fractions to medium wood-waste fractions up to 30 mm in size. The third grinding subunit is configured to grind the medium wood-waste fractions to fine wood-waste fractions up to 6 mm in size. By using such gradual grinding, the wood waste may be properly prepared for the next recycling steps performed by the system.

In one embodiment, the sewage sludge has a humidity of 18% and a fragment size of 2-6 mm. The sewage sludge prepared in this way ensures more efficient operation of the system.

In one embodiment, the sewage sludge storage is equipped with a moving floor. The moving floor may be used to provide a desired metered supply of the sewage sludge to the mixer, which may be beneficial in some use scenarios.

In one embodiment, the system further comprises a sewage sludge silo, a wood waste silo, and a waste mixture silo. The sewage sludge silo is arranged between the sewage sludge storage and the mixer. The wood waste silo is arranged between the grinding unit and the mixer. The waste mixture silo is arranged between the mixer and the metering hopper. In this embodiment, each of the sewage sludge silo, the wood waste silo and the waste mixture silo is configured as a cylindrical top-loaded reservoir comprising a charging bucket conveyor and a discharge screw conveyor. These silos may be used for the long-term storage of the sewage sludge, the wood waste, and the waste mixture, which may be required when the recycling process needs to be suspended (e.g., due to maintenance and/or repair work to be done in the system).

In one embodiment, the metering hopper comprises a screw conveyor, an electric drive, a working chamber, a charging cone, an air outlet nozzle, a carbon dioxide inlet nozzle, and an electric sliding gate. The screw conveyor has a first end and a second end, and the electric drive is coupled to the first end of the screw conveyor to drive the screw conveyor. The working chamber is coupled to the second end of the screw conveyor. The charging cone is attached to the screw conveyor near the first end of the screw conveyor. The air outlet nozzle is attached to the working chamber. The carbon dioxide inlet nozzle is attached to the screw conveyor near the first end of the screw conveyor. The electric sliding gate is attached to the working chamber from below. The electric sliding gate is configured to cause the waste mixture to move from the working chamber to the screw-conveyor dryer. By using the metering hopper thus configured, it is possible to deliver a desired (metered) amount of the waste mixture to the screw-conveyor dryer more efficiently.

In one embodiment, the screw-conveyor dryer comprises a hollow body, a top electric drive, a middle electric drive, a bottom electric drive, and a charging hopper. The hollow body comprises an inlet nozzle, a top coupling pipe, an outlet nozzle, a bottom tube, a bottom coupling pipe, a middle tube, and a top tube. Each of the bottom tube, the middle tube and the top tube has a screw conveyor arranged therein. The inlet nozzle is coupled to a system for supplying a gaseous heat-conducting medium to the hollow body. The outlet nozzle is coupled to a system for removing the gaseous heat-conducting medium from the hollow body. The top coupling pipe is configured to connect the top tube and the middle tube. The bottom coupling pipe is configured to connect the middle tube and the bottom tube. The middle tube is larger than the top tube in diameter but smaller than the bottom tube in diameter. The top electric drive is coupled to the screw conveyor in the top tube. The middle electric drive is coupled to the screw conveyor in the middle tube. The bottom electric drive is coupled to the screw conveyor in the bottom tube. The charging hopper is attached to the top tube and configured to receive the waste mixture from the metering hopper. By using the dryer thus configured, it is possible to heat and dehumidify the waste mixture more efficiently.

In one embodiment, the striker mill comprises a working chamber, a vertical drive shaft, an electric drive, and a horizontal spreading disk. The working chamber has a first cavity and a second cavity. The first cavity has a cylindrical part and a toroidal part. The cylindrical part has a bottom, and the cylindrical part and the toroidal part are interconnected near the bottom of the cylindrical part through an annular slot. The cylindrical part has an inlet pipe coupled to the screw-conveyor dryer and an outlet pipe coupled to the cyclonic separator. The second cavity surrounds the toroidal part of the first cavity and has at least one inlet nozzle for a heat-conducting medium and at least one outlet nozzle for the heat-conducting medium. The vertical drive shaft has a first end arranged outside the working chamber and a second end arranged inside the cylindrical part of the first cavity. The second end of the vertical drive shaft is attached to the horizontal spreading disk which in turn is aligned with the annular slot. The electric drive is coupled to the first end of the vertical drive shaft, and the horizontal spreading disk is attached to the second end of the vertical drive shaft. By using the striker mill thus configured, it is possible to perform the high-temperature thermolysis (thermal destruction) of the waste mixture more efficiently.

Other features and advantages of the present disclosure will be apparent upon reading the following detailed description and reviewing the accompanying drawings.

### BRIEF DESCRIPTION OF THE DRAWINGS

The present disclosure is explained below with reference to the accompanying drawings in which:

FIG. 1 shows a block diagram of a system for conducting high-temperature thermolysis of a waste mixture in accordance with one exemplary embodiment;

FIG. 2 shows a front view of a metering hopper included in the system of FIG. 1 in accordance with one exemplary embodiment;

FIG. 3 shows a front view of a screw-conveyor dryer included in the system of FIG. 1 in accordance with one exemplary embodiment; and

FIG. 4 shows a sectional front view of a striker mill included in the system of FIG. 1 in accordance with one exemplary embodiment.

### DETAILED DESCRIPTION

Various embodiments of the present disclosure are further described in more detail with reference to the accompanying drawings. However, the present disclosure may be embodied in many other forms and should not be construed as limited to any certain structure or function discussed in the following description. In contrast, these embodiments are provided to make the description of the present disclosure detailed and complete.

According to the detailed description, it will be apparent to the ones skilled in the art that the scope of the present disclosure encompasses any embodiment thereof, which is disclosed herein, irrespective of whether this embodiment is implemented independently or in concert with any other embodiment of the present disclosure. For example, the system disclosed herein may be implemented in practice by using any numbers of the embodiments provided herein. Furthermore, it should be understood that any embodiment of the present disclosure may be implemented using one or more of the features presented in the appended claims.

The word “exemplary” is used herein in the meaning of “used as an illustration”. Unless otherwise stated, any embodiment described herein as “exemplary” should not be construed as preferable or having an advantage over other embodiments.

Any positioning terminology, such as “left”, “right”, “top”, “bottom”, “above” “below”, “upper”, “lower”, “horizontal”, “vertical”, “front”, “rear”, etc., may be used herein for convenience to describe one element's or feature's relationship to one or more other elements or features in accordance with the figures. It should be apparent that the positioning terminology is intended to encompass different orientations of the apparatus disclosed herein, in addition to the orientation(s) depicted in the figures. As an example, if one imaginatively rotates the apparatus in the figures 90 degrees clockwise, elements or features described as “left” and “right” relative to other elements or features would then be oriented, respectively, “above” and “below” the other elements or features. Therefore, the positioning terminology used herein should not be construed as any limitation of the invention.

Although the numerative terminology, such as “first”, “second”, etc., may be used herein to describe various embodiments, elements or features, these embodiments, elements or features should not be limited by this numerative terminology. This numerative terminology is used herein only to distinguish one embodiment, element or feature from another embodiment, element or feature. For example, a first thermolysis liquid storage may be called a second thermolysis liquid storage, and vice versa, without departing from the teachings of the present disclosure.

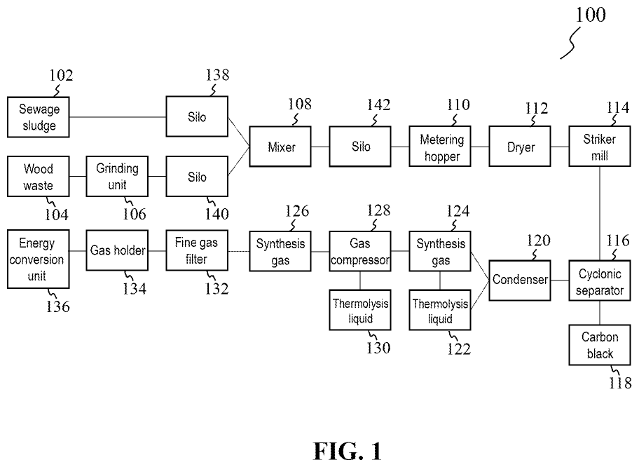

FIG. 1 shows a block diagram of a system 100 for conducting high-temperature thermolysis of a waste mixture in accordance with one exemplary embodiment. The system 100 comprises the following constructive elements: a sewage sludge storage 102, a wood waste storage 104, a grinding unit 106, a mixer 108, a metering hopper 110, a screw-conveyor dryer 112, a striker mill 114, a cyclonic separator 116, a carbon black storage 118, a refrigerator-type condenser 120, a first thermolysis liquid storage 122, a first synthesis gas storage 124, a second synthesis gas storage 126, a gas compressor 128, a second thermolysis liquid storage 130, a fine gas filter 132, a gas holder 134, and an energy conversion unit 136. Each of the constructive elements of the system 100 will be described in detail below.

The sewage sludge storage 102 is configured to receive and store sewage sludge. In the embodiments disclosed herein, the sewage sludge may refer to the residual, semi-solid material that is produced as a by-product during sewage treatment of industrial or municipal wastewater. For example, the sewage sludge may be collected in sewage treatment plants. Preferably, before entering the sewage sludge storage 102, the sewage sludge is pre-treated to have a humidity of 18% and a fragment size of 2-6 mm. The sewage sludge storage 102 may be a production facility having technological equipment for receiving, storing, and accounting for the sewage sludge, as well as removing metals and other (unusual for the sewage sludge) substances from the sewage sludge. The sewage sludge storage 102 may be equipped with a moving floor, which may be required for the constant supply of the sewage sludge. A large volume of the sewage sludge is placed on the moving floor, the moving floor itself may serve as a kind of hopper, but its advantage over a conventional hopper is that it is quite easy to load the moving floor by unloading the sewage sludge from a vehicle body directly onto the moving floor.

The wood waste storage is configured to receive and store wood waste. The wood waste storage 104 may be a production facility having technological equipment for receiving, storing and accounting for the wood waste, as well as removing metals and other (unusual for the wood waste) substances from the wood waste.

The grinding unit 106 is coupled to the wood waste storage 104 and configured to grind the wood waste. To properly prepare the wood waste for further recycling steps, the grinding unit 106 may gradually grind the wood waste. For example, the grinding unit 106 may comprise a coarse-grinding subunit, a medium-grinding subunit and a fine grinding subunit which are arranged in sequence between the wood waste storage 104 and the mixer 108. The coarse-grinding subunit may be configured to grind the wood waste to coarse wood-waste fractions ranging in size from 100 mm to 200 mm. As the coarse-grinding subunit, a single-shaft shredder may be used. The medium-grinding subunit may be configured to grind the coarse wood-waste fractions to medium wood-waste fractions up to 30 mm in size. As the medium-grinding subunit, a twin-shaft shredder may be used. The fine-grinding subunit may be configured to grind the medium wood-waste fractions to fine wood-waste fractions up to 6 mm in size. As the fine-grinding subunit, a fine-grinding mill may be used.

The mixer 108 is configured to form the waste mixture by mixing the sewage sludge from the sewage sludge storage 102 and the grinded wood waste from the grinding unit 106. If the grinding unit 106 comprises the above-indicated three grinding subunits, then the mixer 108 is configured to receive the fine wood-waste fractions. The mixer 108 may be a metal container having a cavity in which there are working bodies connected with a drive and configured to provide mixing of the sewage sludge and the wood waste. The mixer 108 may also be equipped with conveyors for charging the sewage sludge and the wood waste, as well as with a screw conveyor for discharging the resulting waste mixture from the mixer 108 to the outside.

The metering hopper 110 is arranged between the mixer 108 and the screw-conveyor dryer 112. The metering hopper 110 is configured to perform a metered (or dosed) supply of the waste mixture from the mixer 108 to the screw-conveyor dryer 112 while replacing ambient air with carbon dioxide (CO₂).

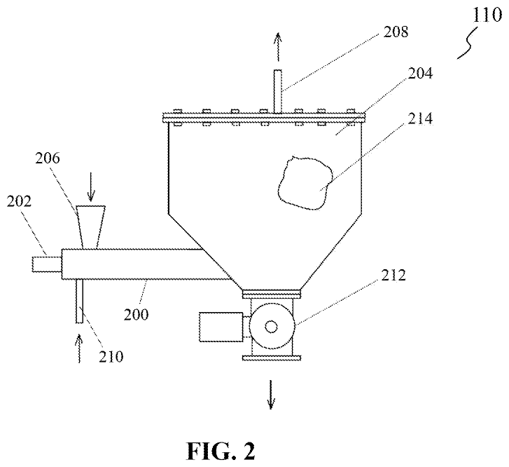

FIG. 2 shows a front view of the metering hopper 110 included in the system 100 in accordance with one exemplary embodiment. As shown in FIG. 2, the metering hopper 110 comprises a screw conveyor 200, an electric drive 202, a working chamber 204, a charging cone 206, an air outlet nozzle 208, a carbon dioxide inlet nozzle 210, and an electric sliding gate 212. The screw conveyor 200 has a first (left) end and a second (right) end, and the electric drive 202 (e.g., a gear motor) is coupled to the first end of the screw conveyor 200 (i.e., to its working body) to drive the working body of the screw conveyor 200. The working chamber 204 is coupled to the second end of the screw conveyor 200 and has an inner cavity 214. The charging cone 206 is attached to the screw conveyor 200 near the first end of the screw conveyor 200 and communicates with the transport cavity of the screw conveyor 200. The air outlet nozzle 208 is attached to the working chamber 204 (i.e., to its upper section) and communicates with the inner cavity 214. The carbon dioxide inlet nozzle 210 is attached to the screw conveyor 200 near the first end of the screw conveyor 200 (i.e., under the charging cone 206) and communicates with the transport cavity of the screw conveyor 200. Thus, the screw conveyor 200 may provide the supply of the waste mixture from the mixer 108 and CO₂ into the inner cavity 214 of the working chamber 204. The electric sliding gate 212 is attached to the working chamber 204 from below. The electric sliding gate 212 is configured to cause the waste mixture to move from the inner cavity 214 to the screw-conveyor dryer 112. The metering hopper 110 thus configured provides the metered or dosed supply of the waste mixture to the screw-conveyor dryer 112, as well as ensures the displacement of environmental air and its replacement with CO₂ directly at the inlet to the screw-conveyor dryer 112.

The screw-conveyor dryer 112 is coupled to the metering hopper 110. The screw-conveyor dryer 112 is configured to heat and dehumidify the waste mixture coming from the metering hopper 110.

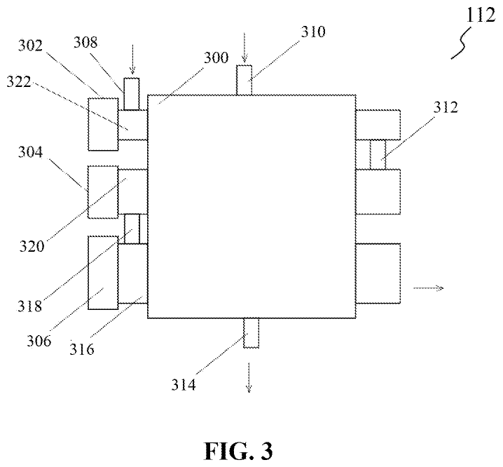

FIG. 3 shows a front view of the screw-conveyor dryer 112 included in the system 100 in accordance with one exemplary embodiment. The screw-conveyor dryer 112 comprises a hollow body 300, a top electric drive 302, a middle electric drive 304, a bottom electric drive 306, and a charging hopper 308. The hollow body 300 comprises an inlet nozzle 310, a top coupling pipe 312, an outlet nozzle 314, a bottom tube 316, a bottom coupling pipe 318, a middle tube 320, and a top tube 322. Each of the bottom tube 316, the middle tube 320 and the top tube 322 has a screw conveyor arranged therein. The top electric drive 302 is coupled to the screw conveyor in the top tube 322. The middle electric drive 304 is coupled to the screw conveyor in the middle tube 320. The bottom electric drive 306 is coupled to the screw conveyor in the bottom tube 316. The charging hopper 308 is attached to the top tube 322 and configured to receive the waste mixture from the metering hopper 110. The inlet nozzle 310 is coupled to a system for supplying a gaseous heat-conducting medium to the cavity of the hollow body 300. The outlet nozzle 314 is coupled to a system for removing the gaseous heat-conducting medium from the cavity of the hollow body 300. The middle sections of the top tube 322, the middle tube 320 and the bottom tube 316 are arranged in the cavity of the hollow body 300 and are in contact with the gaseous heat-conducting medium, while the end sections of the top tube 322, the middle tube 320 and the bottom tube 316 are arranged outside the hollow body 300. The top coupling pipe 312 is configured to connect the top tube 322 and the middle tube 320. The bottom coupling pipe 318 is configured to connect the middle tube 320 and the bottom tube 316. The middle tube 320 is larger than the top tube 322 in diameter but smaller than the bottom tube 316 in diameter. From this circumstance, it follows that with equal productivity of these three sections, the revolutions of the screw conveyor arranged in the through cavity of the middle tube 320 are greater than the revolutions of the screw conveyor arranged in the through cavity of the top tube 322, but less than the revolutions of the screw conveyor arranged in the through cavity of the bottom tube 316. It is these ratios that have a positive effect on the time the transported waste mixture stays in heat transfer zones (this is required to ensure that the waste mixture is evenly heated to a desired temperature), as well as on the quality of the transported waste mixture transportation in terms of preventing the waste mixture from sticking to the contact surfaces.

During the operation of the screw-conveyor dryer 112, thermal energy from the gaseous heat-conducting medium is transferred to the middle sections of the top tube 322, the middle tube 320 and the bottom tube 316, and then to the transported waste mixture. The middle section of at least one of the top tube 322, the middle tube 320 and the bottom tube 316 has holes to remove the water vapor formed during drying. The heat-affected waste mixture from the screw-conveyor dryer 112 is removed from the through cavity of the bottom tube 316 by means of the screw conveyor arranged therein.

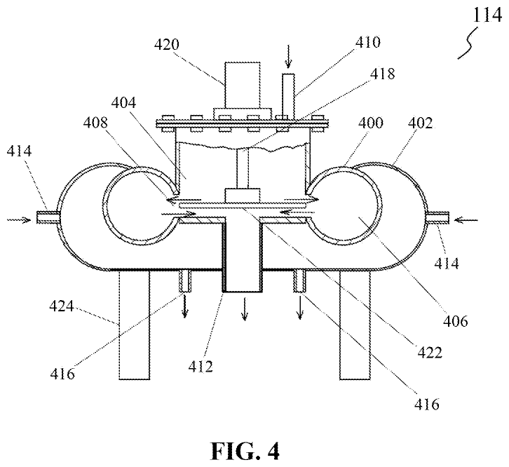

The striker mill 114 is coupled to the screw-conveyor dryer 112. The striker mill 114 is configured to conduct the high-temperature thermolysis of the waste mixture, regrind the waste mixture, and remove the products of the thermolysis outside the striker mill 114. The striker mill 114 provides a preliminary separation of the vapor-gas mixture from solid fractions, which are removed from it in different directions. The striker mill 114 is a multifunctional object, and the implementation of its functions is carried out by means of a single electric drive (like the one denoted by 420 in FIG. 4).

FIG. 4 shows a sectional front view of the striker mill 114 included in the system 100 in accordance with one exemplary embodiment. The striker mill 114 comprises a working chamber comprising a first (inner) cavity 400 and a second (outer) cavity 402. The first cavity 400 has a cylindrical part 404 and a toroidal part 406 which are interconnected near the bottom of the cylindrical part 404 through an annular slot 408. The cylindrical part 404 has an inlet pipe 410 coupled to the screw-conveyor dryer 112 and an outlet pipe 412 coupled to the cyclonic separator 116. The second cavity 402 surrounds the toroidal part 406 of the first cavity 400 and has two inlet nozzles 414 for a (e.g., gaseous) heat-conducting medium and two outlet nozzles 416 for the heat-conducting medium. It should be noted that the present disclosure is not limited to the numbers of the inlet and outlet nozzles 414 and 416 which are shown in FIG. 4; in other embodiments, the second cavity 402 may have a single inlet nozzle and a single outlet nozzle for the heat-conducting medium, or more than two inlet nozzles and more than two outlet nozzles for the heat-conducting medium. The striker mill 114 further comprises a vertical drive shaft 418 having a first end arranged outside the working chamber and a second end arranged inside the cylindrical part 404 of the first cavity 400. The second end of the vertical drive shaft 418 is attached to a horizontal spreading disk 422 which is aligned with the annular slot 408. The striker mill 114 further comprises an electric drive 420 coupled to the first end of the vertical drive shaft 418, while the horizontal spreading disk 422 is attached to the second end of the vertical drive shaft 418. The working chamber may be supported by one or more vertical posts 424.

Through the inlet pipe 410, the waste mixture is fed from the screw-conveyor dryer 112 into the cylindrical part 404 of the first cavity 400 and then to the horizontal spreading disk 422. The waste mixture is fed into the toroidal part 406 of the first cavity 400 due to the centrifugal force exerting on the waste mixture arranged on the surface of the horizontal spreading disk 422, as well as due to the fact that the second end of the vertical drive shaft 418 is attached to the horizontal spreading disk 422 aligned with the annular slot 408. The horizontal spreading disk 422 is installed relative to the annular groove 408 such that the waste mixture is fed into the toroidal part 406 of the first cavity 400 to conduct the high-temperature thermolysis (or thermal destruction) exclusively through the upper section of the annular groove 408, and the products of the high-temperature thermolysis are removed from the toroidal part 406 of the first cavity 400 exclusively through the lower section of the annular groove 408 and the outlet pipe 412 (this is schematically shown by means of arrows in FIG. 4). The temperature required for the high-temperature thermolysis is provided in the toroidal part 406 of the first cavity 400 by supplying the heat-conducting medium, through the inlet nozzles 414, into the second cavity 402. The heat-conducting medium is then removed from the second cavity 402 through the outlet nozzles 416.

The operational principle of the striker mill 114 is as follows.

The temperature required for the high-temperature thermolysis is provided and maintained in the second cavity 402 (including its wall) and in the toroidal part 406 (including its wall) of the first cavity 400 throughout the high-temperature thermolysis. After that, the electric drive 420 is turned on, thereby causing the vertical drive shaft 418 and, consequently, the spreading horizontal disc 422 to rotate. Then, the waste mixture is fed from the screw-conveyor dryer 112 into the cylindrical part 404 of the first cavity 400. Under the influence of the centrifugal force, the waste mixture arranged on the surface of the horizontal spreading disk 422 moves into the toroidal part 406 of the first cavity 400, hits the hot section of the wall of the toroidal part 406, whereupon it heats up and mechanically breaks into numerous fragments, thereby leading to a significant increase in the area of the fragments of the waste mixture by 20-50 times. The presence of the concave section of the wall of the toroidal part 406 provides numerous collisions of the fragments of the waste mixture against the concave section of the wall of the toroidal part 406, as well as between themselves, which leads to their additional heating and grinding. The rotating horizontal spreading disk 422 forms a vortex flow that supplies the waste mixture through the upper section of the annular slot 408 into the toroidal part 406 and subsequently removes the products of the high-temperature thermolysis from the toroidal part 406 of the first cavity 400 through the lower section of the annular slot 408 and the outlet pipe 412 (see the arrows in FIG. 4). These products comprise a vapor-gas mixture and a solid fraction.

The cyclonic separator 116 is coupled to the striker mill 114 and configured to separate the waste mixture subjected to the high-temperature thermolysis and regrinding into a solid fraction and a gaseous (gas-vapor) fraction. The solid fraction of the waste mixture comprises carbon black.

The carbon black storage 118 is coupled to the cyclonic separator 116 and configured to receive and store the carbon black from the waste mixture. The carbon black storage 118 may be a container having a vertical side wall, a conical bottom with a gate valve and an electric drive, by means of which the extraction of a solid dispersed fraction in the form of carbon black from the storage 118 is ensured.

The refrigerator-type condenser 120 is coupled to the cyclonic separator 116 and configured to obtain a thermolysis liquid and a synthesis gas by cooling and condensing the gaseous fraction of the waste mixture. The refrigerator-type condenser 120 may be a heat exchanger in which the following processes are performed: a condensation process, and a process of phase transition of a heat-conducting medium from a vapor-gas state to a liquid state due to heat removal by using a coolant.

The superheated vapor-gas mixture enters the refrigerator-type condenser 120, which is cooled to its saturation temperature and, condensing, passes into a liquid phase with the formation of the thermolysis liquid and the synthesis gas. To condense steam, it is necessary to remove, from each unit of its mass, heat equal to a specific heat of condensation. The refrigerator-type condenser 120 may be of water-cooled type.

The first thermolysis liquid storage 122 is coupled to the refrigerator-type condenser 120 and configured to receive and store the thermolysis liquid. The storage 122 may comprise a system for supplying and removing the thermolysis liquid.

The first synthesis gas storage 124 is coupled to the refrigerator-type condenser 120 and configured to receive and store the synthesis gas. The storage 124 is stationary and has a constant volume. In the cavity of the storage 124, the synthesis gas is under pressure of 1 atm. The storage 124 comprises a pump and a gas burner with an emergency valve (emergency torch).

The gas compressor 128 is arranged between the first synthesis gas storage 124 and the second synthesis gas storage 126. The gas compressor 128 is configured to pump over the synthesis gas from the first synthesis gas storage 124 to the second synthesis gas storage 126 while increasing a pressure of the synthesis gas up to 12 atm.

The second thermolysis liquid storage 130 is coupled to the gas compressor 128 and configured to collect and store a secondary thermolysis liquid resulted from compressing the synthesis gas by the gas compressor 128 when pumping over the synthesis gas from the first synthesis gas storage 124 to the second synthesis gas storage 126.

The fine gas filter 132 is coupled to the second synthesis gas storage 126 and configured to clean the synthesis gas from mechanical impurities.

The gas holder 134 is coupled to the fine gas filter 132 and configured to receive and store the (cleaned) synthesis gas under pressure of up to 30 atm. The gas holder 134 may comprise a high pressure compressor and a reducer for lowering and stabilizing the low pressure of the synthesis gas leaving the cavity of the gas holder 134.

The energy conversion unit 136 is coupled to the gas holder 134 and configured to convert the chemical energy of the pressurized synthesis gas into thermal energy and electrical energy. The energy conversion unit 136 may be implemented as a gas-piston power plant comprising an internal combustion engine and a thermal energy extraction system; such a plant provides the conversion of the chemical energy of the burned synthesis gas into thermal and electrical energy. The dominant part of the thermal energy generated during the combustion of the synthesis gas converts into a liquid that cools a piston group of the internal combustion engine, and is also removed, outside the internal combustion engine, with exhaust gases. To extract the thermal energy from the heated liquid, a heat exchanger is used, where the thermal energy is transferred from a liquid with a higher temperature to a liquid with a lower temperature. To extract the thermal energy from hot exhaust gases, a heat exchanger is also used, where thermal energy is transferred from hot exhaust gases to a heat-conducting liquid having a lower temperature. The chemical energy of the burned synthesis gas is converted into electrical energy by means of a generator kinematically connected to a rotating shaft of the internal combustion engine.

Referring back to FIG. 1, the system 100 may optionally comprise a sewage sludge silo 138, a wood waste silo 140, and a waste mixture silo 142. The sewage sludge silo 138 is arranged between the sewage sludge storage 102 and the mixer 108. The wood waste silo 140 is arranged between the grinding unit 106 and the mixer 108. The waste mixture silo 142 is arranged between the mixer 108 and the metering hopper 112. In this embodiment, each of the sewage sludge silo 138, the wood waste silo 140 and the waste mixture silo 142 is configured as a cylindrical top-loaded reservoir comprising a charging bucket conveyor and a discharge screw conveyor.

### EXPERIMENT

The constructive elements of the system 100 was arranged in the order shown in FIG. 1. As raw materials, the sewage sludge pre-cleaned (from mechanical impurities) and pre-treated to have a humidity of 18% and a fragment size of 2-6 mm, as well as creosote-impregnated fragments of wooden railway sleepers and utility poles (with the same parameters as indicated above) were used.

A waste mixture was obtained, which comprises 55 wt. % of sewage sludge and 45 wt. % of wood waste. When conducting a practical experiment, 1000 kg of the waste mixture were used. The experiment was performed in a carbon dioxide (CO₂) environment without access to ambient air, which is necessary to ensure the safety of the process, as well as to exclude oxidative processes between the products formed during the thermolysis of the waste mixture. The thermolysis of the waste mixture was performed at a temperature of 780 to 820° C. in the toroidal part 406 of the first cavity 400 in the striker mill 114. The displacement of ambient air and its replacement with carbon dioxide was performed by means of the metering hopper 110. The waste mixture was subjected to dehumidification by means of the screw-conveyor dryer 112.

As a result of the experiment, thermal energy, electrical energy, a liquid fraction in the form of a thermolysis liquid, as well as a synthesis gas and carbon black were obtained. The synthesis gas was used to obtain the thermal energy and the electrical energy.

The gas chromatographic analysis of the synthesis gas obtained by the thermolysis of the waste mixture showed the presence of the following components in the synthesis gas:

| | vol. % | | | | | | | | | | | |
|---|---|---|---|---|---|---|---|---|---|---|---|---|
| Component | | | | | | Ethane | Propane | C4 | C5 | C6 | | Total |
| name | H2 | N2 | CH4 | CO | CO2 | Ethylene | Propylene | sum | sum | sum | H2O | sum |
| 780° C.- | 36.70 | 10.15 | 16.97 | 22.90 | 6.90 | 2.88 | 0.12 | 0.10 | 0.68 | 0.38 | 2.30 | 100.0 |
| 820° C. | | | | | | | | | | | | |

The calorific value of the synthesis gas (by combustible fraction): Qn lower = 16.45 MJ/m³.
The density of the synthesis gas: P = 0.93 kg/m³.

The resulting synthesis gas was fed into the internal combustion engine of a gas-piston power plant, where the chemical energy of the synthesis gas was converted into thermal energy and electrical energy. Carbon black was obtained at the outlet of the cyclonic separator 116 and stored in the carbon black storage 118. The thermolysis liquid entered the first thermolysis liquid storage 122 and the second thermolysis liquid storage 130.

Although the exemplary embodiments of the present disclosure are described herein, it should be noted that any various changes and modifications could be made in the embodiments of the present disclosure, without departing from the scope of legal protection which is defined by the appended claims. In the appended claims, the word “comprising” does not exclude other elements or operations, and the indefinite article “a” or “an” does not exclude a plurality. The mere fact that certain measures are recited in mutually different dependent claims does not indicate that a combination of these measures cannot be used to advantage.

1. A system for conducting high-temperature thermolysis of a waste mixture, comprising:
a sewage sludge storage configured to receive and store sewage sludge;
a wood waste storage configured to receive and store wood waste;
a grinding unit coupled to the wood waste storage and configured to grind the wood waste;
a mixer configured to form the waste mixture by mixing the sewage sludge from the sewage sludge storage and the grinded wood waste from the grinding unit;
a screw-conveyor dryer configured to heat and dehumidify the waste mixture;
a metering hopper arranged between the mixer and the screw-conveyor dryer, the metering hopper being configured to perform a metered supply of the waste mixture from the mixer to the screw-conveyor dryer while replacing ambient air with carbon dioxide (CO₂);
a striker mill coupled to the screw-conveyor dryer (9) and configured to conduct the high-temperature thermolysis of the waste mixture and regrind the waste mixture;
a cyclonic separator coupled to the striker mill and configured to separate the waste mixture subjected to the high-temperature thermolysis and regrinding into a solid fraction and a gaseous fraction, the solid fraction of the waste mixture comprising carbon black;
a carbon black storage coupled to the cyclonic separator and configured to receive and store the carbon black;
a refrigerator-type condenser coupled to the cyclonic separator and configured to obtain a thermolysis liquid and a synthesis gas by cooling and condensing the gaseous fraction of the waste mixture;
a first thermolysis liquid storage coupled to the refrigerator-type condenser and configured to receive and store the thermolysis liquid;
a first synthesis gas storage coupled to the refrigerator-type condenser and configured to receive and store the synthesis gas;
a second synthesis gas storage coupled to the first synthesis gas storage;
a gas compressor arranged between the first synthesis gas storage and the second synthesis gas storage, the gas compressor being configured to pump over the synthesis gas from the first synthesis gas storage to the second synthesis gas storage while increasing a pressure of the synthesis gas up to 12 atm;
a second thermolysis liquid storage coupled to the gas compressor and configured to collect and store a secondary thermolysis liquid resulted from compressing the synthesis gas by the gas compressor when pumping over the synthesis gas from the first synthesis gas storage to the second synthesis gas storage;
a fine gas filter coupled to the second synthesis gas storage and configured to clean the synthesis gas from mechanical impurities;
a gas holder coupled to the fine gas filter and configured to receive and store the synthesis gas under pressure of up to 30 atm; and
an energy conversion unit coupled to the gas holder and configured to convert chemical energy stored in the pressurized synthesis gas into thermal energy and electrical energy.

2. The system of claim 1, wherein the grinding unit comprises:
a first grinding subunit configured to grind the wood waste to coarse wood-waste fractions ranging in size from 100 mm to 200 mm;
a second grinding subunit configured to grind the coarse wood-waste fractions to medium wood-waste fractions up to 30 mm in size; and
a third grinding subunit configured to grind the medium wood-waste fractions to fine wood-waste fractions up to 6 mm in size.

3. The system of claim 1, wherein the sewage sludge has a humidity of 18% and a fragment size of 2-6 mm.

4. The system of claim 1, wherein the sewage sludge storage is equipped with a moving floor.

5. The system of claim 1, further comprising:
a sewage sludge silo arranged between the sewage sludge storage and the mixer;
a wood waste silo arranged between the grinding unit and the mixer; and
a waste mixture silo arranged between the mixer and the metering hopper;
wherein each of the sewage sludge silo, the wood waste silo and the waste mixture silo is configured as a cylindrical top-loaded reservoir comprising a charging bucket conveyor and a discharge screw conveyor.

6. The system of claim 1, wherein the metering hopper comprises:
a screw conveyor having a first end and a second end;
an electric drive coupled to the first end of the screw conveyor, the electric drive being configured to drive the screw conveyor;
a working chamber coupled to the second end of the screw conveyor;
a charging cone attached to the screw conveyor near the first end of the screw conveyor;
an air outlet nozzle attached to the working chamber;
a carbon dioxide inlet nozzle attached to the screw conveyor near the first end of the screw conveyor; and
an electric sliding gate attached to the working chamber from below, the electric sliding gate being configured to cause the waste mixture to move from the working chamber to the screw-conveyor dryer.

7. The system of claim 1, wherein the screw-conveyor dryer comprises:
a hollow body comprising an inlet nozzle, a top coupling pipe, an outlet nozzle, a bottom tube, a bottom coupling pipe, a middle tube, and a top tube, wherein each of the bottom tube, the middle tube and the top tube having a screw conveyor arranged therein, the inlet nozzle is coupled to a system for supplying a gaseous heat-conducting medium to the hollow body, the outlet nozzle is coupled to a system for removing the gaseous heat-conducting medium from the hollow body, the top coupling pipe is configured to connect the top tube and the middle tube, the bottom coupling pipe is configured to connect the middle tube and the bottom tube, and the middle tube is larger than the top tube in diameter but smaller than the bottom tube in diameter;
a top electric drive coupled to the screw conveyor in the top tube;
a middle electric drive coupled to the screw conveyor in the middle tube;
a bottom electric drive coupled to the screw conveyor in the bottom tube; and
a charging hopper attached to the top tube and configured to receive the waste mixture from the metering hopper.

8. The system of claim 1, wherein the striker mill comprises:
a working chamber having a first cavity and a second cavity, the first cavity having a cylindrical part and a toroidal part, the cylindrical part having a bottom, the cylindrical part and the toroidal part being interconnected near the bottom of the cylindrical part through an annular slot, the cylindrical part having an inlet pipe coupled to the screw-conveyor dryer and an outlet pipe coupled to the cyclonic separator, the second cavity surrounding the toroidal part of the first cavity, the second cavity having at least one inlet nozzle for a heat-conducting medium and at least one outlet nozzle for the heat-conducting medium;
a vertical drive shaft having a first end arranged outside the working chamber and a second end arranged inside the cylindrical part of the first cavity;
an electric drive coupled to the first end of the vertical drive shaft; and
a horizontal spreading disk attached to the second end of the vertical drive shaft, the horizontal spreading disk being arranged opposite to and aligned with the annular slot, the horizontal spreading disk being configured to feed the waste mixture into the toroidal part of the first cavity through an upper section of the annular slot and to remove the waste mixture subjected to the high-temperature thermolysis and regrinding from the toroidal part of the first cavity through a lower section of the annular slot.
